(19) United States
(12) Patent Application Publication
(10) Pub. No.: US 2017/0120018 A1
(43) Pub. Date: May 4, 2017

(54) GUIDEWIRE

(71) Applicant: TERUMO KABUSHIKI KAISHA, Tokyo (JP)

(72) Inventors: Yasunao OOTANI, Ito-shi (JP); Yutaka TANO, Fujinomiya-shi (JP)

(73) Assignee: TERUMO KABUSHIKI KAISHA, Tokyo (JP)

(21) Appl. No.: 15/404,647

(22) Filed: Jan. 12, 2017

Related U.S. Application Data
(63) Continuation of application No. PCT/JP2015/074301, filed on Aug. 27, 2015.

(30) Foreign Application Priority Data
Sep. 25, 2014 (JP) 2014-195751

(51) Int. Cl. A61M 25/09

(52) U.S. Cl. CPC A61M 25/09; A61M 2025/09083; A61M 2025/09133; A61M 2025/09166

(57) ABSTRACT

A guidewire is disclosed, which includes a core member that is an elongated member having flexibility, and a coil member that is disposed so as to cover a distal portion of the core member and that is formed by helically winding a strand, the coil member being fixed to the core member in a distal portion of the guidewire. The core member includes a body portion in a proximal portion thereof and a flat portion in the distal portion thereof. At least one projection is formed on at least one of two side surfaces of the flat portion extending in a longitudinal direction, the at least one projection projecting into a space between adjacent turns of the strand and being in contact with the strand.

### CROSS-REFERENCES TO RELATED APPLICATIONS

This application is a continuation of International Application No. PCT/JP2015/074301 filed on Aug. 27, 2015, and which claims priority to Japanese Patent Application No. 2014-195751 filed on Sep. 25, 2014, the entire contents of both, which are incorporated herein by reference.

### TECHNICAL FIELD

The present disclosure relates to a guidewire that is used to guide a catheter into a body lumen, in particular, into a blood vessel.

### BACKGROUND ART

Guidewires are used to perform treatment, such as PTCA (Percutaneous Transluminal Coronary Angioplasty), of a lesion where it is difficult to perform a surgical operation; to perform treatment that is intended to be minimally invasive to the human body; or to guide a catheter used for a medical examination, such as angiocardiography, into a blood vessel. PTCA is a treatment method for improving blood flow in a coronary artery by expanding a stenotic portion of the coronary artery by using a balloon.

In PTCA, in a state in which a distal portion of a guidewire protrudes from the distal end of a balloon catheter, the guidewire is inserted into a blood vessel until the guidewire reaches a position near a stenotic portion of the blood vessel, and thereby the balloon catheter is guided to the stenotic portion. In this process, the guidewire needs to selectively pass through a meandering or branched blood vessel, or a narrowed blood vessel. Accordingly, a PTCA guidewire should have high flexibility (blood vessel trackability) so that the guidewire can easily conform to the shape of a blood vessel to avoid damaging the wall of the blood vessel. Moreover, a PTCA guidewire should have high torque transmission ability, because it is necessary to efficiently transmit rotation of a proximal portion (base end portion) to a distal portion of the guidewire and to change the direction of the distal portion.

In some PTCA cases, in order to help enable a guidewire to conform to a bent or branched blood vessel, shaping of a distal portion of the guidewire may be performed before inserting the guidewire into the blood vessel. For example, a doctor shapes a distal portion of the guidewire by bending the distal portion into a predetermined shape (for example, a J-shape) with his/her fingers in accordance with the shape of a branched blood vessel or the like. Accordingly, it is also required for a guidewire that such shaping of the distal portion can be performed easily.

An example of exiting PTCA guidewires, which has the following structure, is described in International Publication No. WO/2009/126656. The guidewire of International Publication No. WO/2009/126656 includes a core member that is an elongated member and a coil that is disposed so as to cover a distal portion of the core member. The distal portion of the core member includes a flat portion having a width twice or more as large as a height (thickness).

The guidewire of International Publication No. WO/2009/126656 has a flexible distal portion, because the distal portion of the core member of the guidewire includes the flat portion having a small thickness. Therefore, the safety and the blood vessel trackability of the guidewire may be improved to some extent.

However, the flat portion becomes twisted when a proximal portion of such a guidewire is rotated to pass the guidewire through a meandering or branched blood vessel, or a narrowed blood vessel. As a result, a torque applied to the proximal portion of the guidewire is not effectively transmitted to the distal portion, and the distal portion of the guidewire is not directed toward an intended direction. Therefore, the guidewire has a problem in that the torque transmission ability (trackability) is reduced.

### SUMMARY OF INVENTION

A guidewire is disclosed that has high torque transmission ability (trackability: an ability of transferring a torque applied to a proximal portion of the guidewire to a distal portion of the guidewire).

In accordance with an exemplary embodiment, a guidewire according to the present disclosure includes a core member that is an elongated member having flexibility; and a coil member that is disposed so as to cover a distal portion of the core member and that is formed by helically winding a strand, the coil member being fixed to the core member in a distal portion of the guidewire. The core member includes a body portion in a proximal portion thereof and a flat portion in the distal portion thereof. At least one projection is formed on at least one of two side surfaces of the flat portion extending in a longitudinal direction, the at least one projection projecting into a space between adjacent turns of the strand and being in contact with the strand. In the guidewire according to the present disclosure, preferably, a longitudinal sectional shape of the projection is substantially triangular. In the guidewire according to the present disclosure, preferably, a plurality of the projections are formed on each of the two side surfaces of the flat portion extending in the longitudinal direction, and a width of the flat portion including the projections is larger than an inside diameter of the coil member.

A guidewire is disclosed comprising: a core member that is an elongated member having flexibility; a coil member configured to cover a distal portion of the core member and that is formed by helically winding a strand, the coil member being fixed to the core member in a distal portion of the guidewire, wherein the core member includes a body portion in a proximal portion of the core member and a flat portion in the distal portion of the core member; and a plurality of projections formed on each side surface of the flat portion extending in the longitudinal direction, the plurality of projections projecting into a space between adjacent turns of the strand and being in contact with the strand.

With the structure described above, since the core member includes the flat portion in the distal portion thereof, the flexibility of the distal portion of the core member can be improved, and therefore the flexibility of the distal portion of the guidewire can be improved. Moreover, since the flexibility of the distal portion of the guidewire can be improved, shaping of the distal portion of the guidewire can be performed relatively easily in accordance with the shape of a branched blood vessel. Furthermore, since the projection is formed on the side surface of the flat portion extending in the longitudinal direction, frictional resistance acts on the projection, which is in contact with the strand, when the proximal portion of the guidewire is rotated. Accordingly, twisting of the flat portion can be suppressed. As a result, a torque applied to the proximal portion of the guidewire can be effectively transmitted to the distal portion, and the distal portion of the guidewire can be directed toward an intended direction.

With the guidewire according to the present disclosure, the flexibility of the distal portion of the guidewire can be improved, and shaping of the distal portion is of the guidewire can be performed relatively easily. Therefore, the blood vessel trackability of the guidewire can be improved. Moreover, since twisting of the flat portion of the core member can be suppressed, a torque applied to the proximal portion of the guidewire can be effectively transmitted to the distal portion of the guidewire. Therefore, the torque transmission ability of the guidewire can be improved.

### BRIEF DESCRIPTION OF THE DRAWINGS

FIG. 1 is a partial longitudinal sectional view of a guidewire according to an exemplary embodiment of the present disclosure.

FIG. 2 is a partial longitudinal sectional view of a distal portion of the guidewire, taken along a plane at an angle different from that of FIG. 1.

FIG. 3 is a partial longitudinal sectional view in which a part of FIG. 2 is enlarged.

FIG. 4 is an end view of a core member taken along line IV-IV shown in FIG. 2.

FIG. 5 is a sectional view of the core member taken along line V-V shown in FIG. 2.

FIG. 6 is a sectional view illustrating a modification of the core member shown in FIG. 5.

### DETAILED DESCRIPTION

A guidewire according to a first exemplary embodiment of the present disclosure will be described in detail with reference to the drawings. In the present disclosure, a distal portion of the guidewire refers to a portion of the guidewire that is inserted into a blood vessel, and a proximal portion of the guidewire refers to a portion of the guidewire with which, for example, a doctor operates the guidewire.

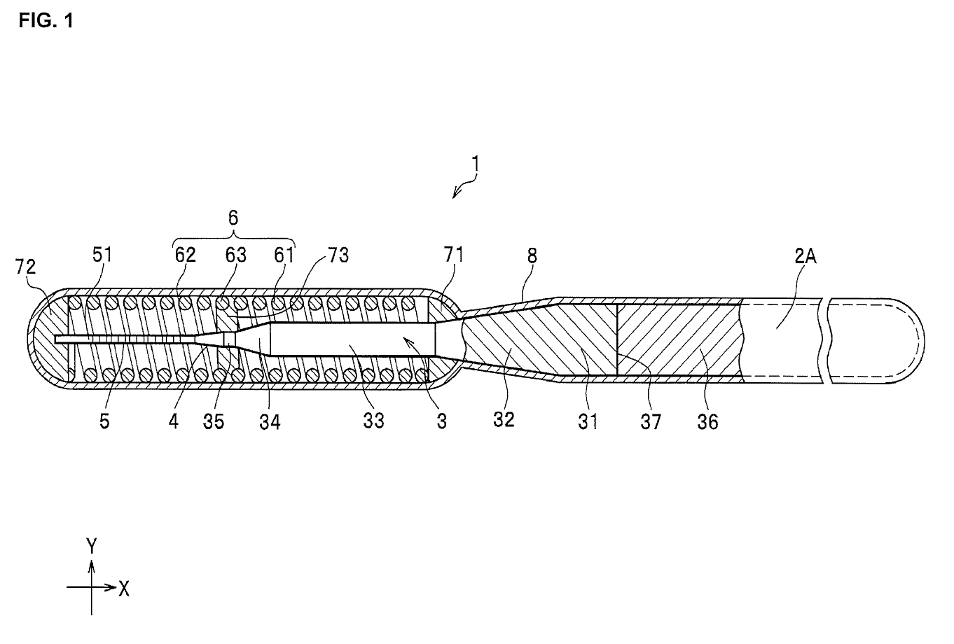

As illustrated in FIG. 1, a guidewire (hereinafter, referred to as a “wire”) 1 is an elongated wire including a core member 2A and a coil member 6. The core member 2A and the coil member 6 are fixed to each other in the distal portion of the wire 1. The core member 2A can include a body portion 3 and a flat portion 5. At least one projection 51 is formed on the flat portion 5. The length of the wire 1 is not particularly limited. Preferably, the wire 1 has a length of, for example, 200 mm to 5000 mm. Preferably, the core member 2A and the coil member 6 are fixed to each other by using a fixing material (fixing portion) 72, such as a solder (brazing alloy) or an adhesive. However, the fixing portion 72 may be formed by welding. Hereinafter, each of the elements of the wire 1 will be described.

As illustrated in FIG. 1, the coil member 6 is a coil that is disposed so as to cover a distal portion of the core member 2A and that is formed by helically winding a strand 6a. The coil may be a so-called “closely-wound coil”, in which adjacent turns of the strand 6a are in contact with each other. Alternatively, the coil may be a coil in which adjacent turns of the strand 6a are separated from each other. A distal portion of the coil member 6 is fixed to the core member 2A (the flat portion 5) by using the fixing material (fixing portion) 72.

The material of the strand 6a is not particularly limited. Preferably, the strand 6a is made of a metal material, for example, such as a stainless steel or a Pt—Ni alloy. The size of the coil member 6 is not particularly limited and may vary depending on the intended use of the wire 1. When the wire 1 is a PTCA guidewire, the strand 6a preferably has a diameter of, for example, 0.01 mm to 0.1 mm, and the coil member 6 preferably has an outside diameter of, for example, 0.2 mm to 0.5 mm and a length of, for example, 10 mm to 1000 mm. Preferably, the outside diameter of the coil member 6 is uniform in the longitudinal direction of the wire 1. However, the outside diameter may decrease toward the distal end of the wire 1.

The coil member 6 may be made of a combination of two or more metal materials. For example, the coil member 6 may include a first coil member 61 that is disposed in a proximal portion of the coil member 6 and that is made from a strand of a stainless steel; a second coil member 62 that is disposed in a distal portion of the coil member 6 and that is made from a strand of a Pt—Ni alloy, which is a radiopaque material; and a boundary portion 63 that is disposed between the first coil member 61 and the second coil member 62 and at which the first and second coil members 61 and 62 are joined to each other by welding, or by using an adhesive. In this case, the distal portion of the wire 1 can be easily seen under X-ray fluoroscopy.

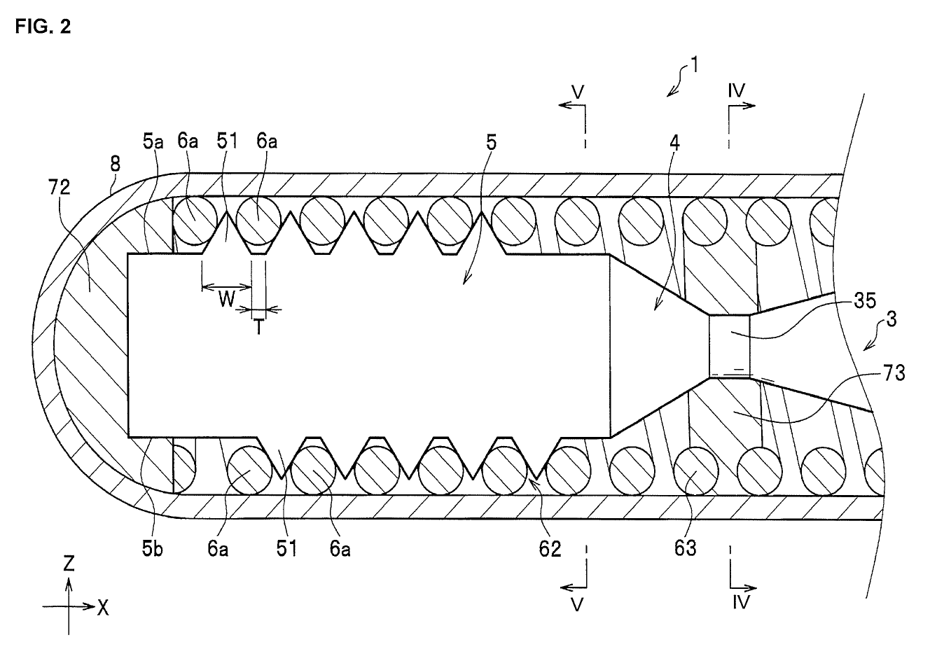

As illustrated in FIGS. 1 and 2, the core member 2A is an elongated member having flexibility. In consideration of the flexibility and strength of the wire 1, preferably, the core member 2A is made of an elastic metal material, such as a Ni—Ti alloy or a stainless steel. The core member 2A can include the body portion 3, a transition portion 4, and the flat portion 5, from the proximal end toward the distal end. At least one projection 51 is formed on the flat portion 5. The core member 2A need not include the transition portion 4.

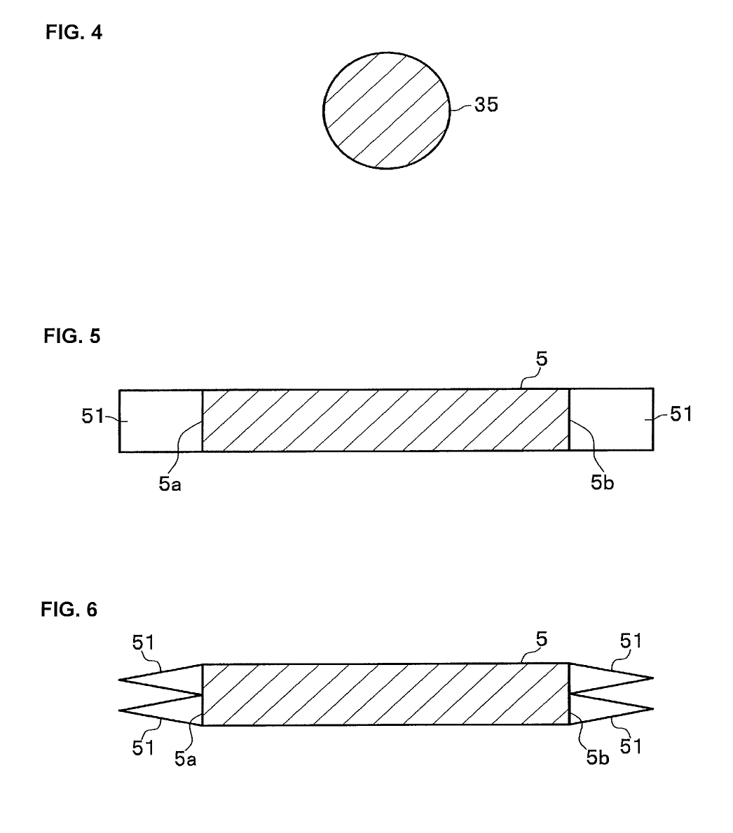

As illustrated in FIGS. 1 and 2, the body portion 3 is an elongated portion having a rod-like shape (non-flat shape). Preferably, the cross-sectional shape of the body portion 3 (taken along a plane parallel to the YZ-plane and perpendicular to the longitudinal direction) is substantially circular (see FIG. 4). Preferably, the body portion 3 can include, from the proximal end toward the distal end, a large-diameter portion 31 having a uniform outside diameter, a first tapered portion 32 having an outside diameter that gradually decreases toward the distal end, an medium-diameter portion 33 having a uniform outside diameter, a second tapered portion 34 having an outside diameter that gradually decreases toward the distal end, and a small-diameter portion 35 having a uniform outside diameter.

In the example described above, the body portion 3 has two tapered portions, which are the first tapered portion 32 and the second tapered portion 34, between the portions having uniform outside diameters (between the large-diameter portion 31 and the medium-diameter portion 33 and between the medium-diameter portion 33 and the small-diameter portion 35). However, the number of tapered portions is not limited to two and may be at least one. A large-diameter portion 36, which has the same outside diameter as the large-diameter portion 31 and which is made of a material different from that of the large-diameter portion 31, may be joined to the large-diameter portion 31 at a joint portion (welded portion) 37. The joining method is not particularly limited. Examples of the joining method include frictional resistance welding, laser spot welding, butt resistance welding such as upset welding, and a joining method using a tubular joint member.

As illustrated in FIGS. 1 and 2, the transition portion 4 connects the body portion 3 and the flat portion 5 to each other. In accordance with an exemplary embodiment, the cross-sectional shape of the transition portion 4 gradually changes from a circle (see FIG. 4) or the like to a rectangle (see FIG. 5) from the proximal end toward the distal end. Preferably, the transition portion 4 has a length of, for example, 1 mm to 10 mm. Preferably, the transition portion 4 is formed together with the flat portion 5 by pressing a distal end portion of the body portion 3, whose cross-sectional shape is a circle and whose diameter is preferably reduced, by using, for example, a die.

As illustrated in FIGS. 1 and 2, the flat portion 5 is an elongated flat plate having a rectangular cross-sectional shape (see FIG. 5) so that the wire 1 (the core member 2A) can have flexibility and shaping of the distal portion of the wire can be performed relatively easily. Preferably, for example, the flat portion 5 has a length of 1 mm to 30 mm, a width of 0.1 mm to 0.5 mm, and a thickness of 0.01 mm to 0.06 mm. The width of the flat portion 5 may increase or decrease toward the distal end. The thickness of the flat portion 5 may also increase or decrease toward the distal end. A distal end portion of the flat portion 5 is fixed to the coil member 6, for example, by using the fixing material (fixing portion) 72.

At least one projection 51 is formed on at least one of two side surfaces of the flat portion 5 extending in the longitudinal direction. Here, a “side surface” of the flat portion 5 is a surface extending in a direction in which the flat portion 5 becomes curved when the wire 1 is used. To be specific, the at least one projection 51 is formed on at least one of a side surface 5a, which is at one end of the flat portion 5 in the width direction, and a side surface 5b, which is at the other end of the flat portion 5 in the width direction. Preferably, a plurality of projections 51 are formed on each of the side surfaces 5a and 5b. A plurality of projections 51 may be formed so as to be arranged not only in the longitudinal direction of the side surfaces 5a and 5b as illustrated in FIG. 2 but also in the height direction of the side surfaces 5a and 5b as illustrated in FIG. 6. When forming a plurality of projections 51 so as to be arranged in the longitudinal direction of the flat portion 5 as illustrated in FIG. 2, preferably, the projections 51 are arranged along the entire length of the flat portion 5. However, the projections 51 may be formed in such a way that a larger number of the projections 51 are disposed in one of the proximal portion and in the distal portion than in the other (not shown). Preferably, the projection 51 is formed by performing machining, such as press forming or laser forming, of the flat portion 5.

As illustrated in FIG. 2, the projections 51 are formed on the side surfaces 5a and 5b of the flat portion 5 at positions such that each of the projections 51 projects into a space between adjacent turns of the strand 6a of the coil member 6, which is helically wound, and is in contact with the strand 6a. Preferably, each of the projections 51 projects into the coil member 6, whose adjacent turns of the strand 6a are separated from each other and is in contact with the strand 6a. However, each of the projections 51 may project into a coil member 6 that is a so-called “closely-wound coil”, whose adjacent turns of the strand 6a are in contact with each other, and be in contact with the strand 6a in a state in which each of the projections 51 separates adjacent turns of the strand 6a from each other.

The longitudinal sectional shape of each of the projections 51 (taken along a plane parallel to the XZ-plane and extending in the longitudinal direction) is not particularly limited, as long as frictional resistance is generated between the projection 51 and the strand 6a. Preferably, the longitudinal sectional shape is a substantially triangular shape as illustrated in FIG. 2. The longitudinal sectional shape may be another shape (not shown), such as a substantially semicircular shape or a substantially rectangular shape. The cross-sectional shape of each of the projections 51 (taken along a plane parallel to the YZ-plane and perpendicular to the longitudinal direction) is not particularly limited, as long as frictional resistance is generated between the projection 51 and the strand 6a. Preferably, the cross-sectional shape is a substantially rectangular shape as illustrated in FIG. 5. The cross-sectional shape may be a substantially triangular shape (see FIG. 6) or another shape (not shown), such as a substantially semicircular shape.

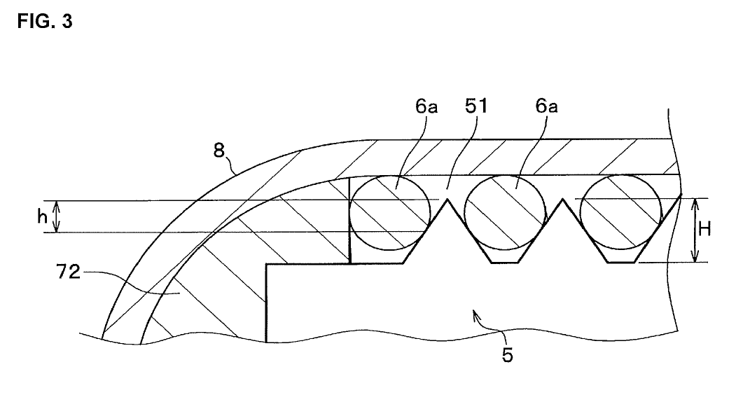

As illustrated in FIGS. 2 and 3, the size of each of the projections 51 is set to such a size that frictional resistance is generated between the projection 51 and the strand 6a. In a case where a plurality of projections 51 are formed, in the example illustrated in FIG. 2, each of the projections 51 projects into a space between adjacent turns of the strand 6a and is in contact with the strand 6a. Alternatively, although not illustrated, two or more of the projections 51 may project into a space between adjacent turns of the strand 6a and be in contact with the strand 6a. In a case where a plurality of projections 51 are formed on each of the two side surfaces of the flat portion 5 extending in the longitudinal direction, the width of the flat portion 5, including the projections 51, is larger than the inside diameter of the coil member 6. In this case, the width of the flat portion 5 and the height H of each of the projections 51 may be appropriately set in accordance with the inside diameter of the coil member 6. In the example illustrated in FIG. 2, each of the projections 51 projects into a space between adjacent turns of the strand 6a and is in contact with the strand 6a. Alternatively, although not illustrated, each of the projections 51 may project into a space between adjacent sets of two or more turns of the strand 6a and be in contact with the strand 6a. Accordingly, for example, each of the projections 51 preferably has a width W of 0.001 mm to 15 mm and a height H of 0.005 mm to 0.15 mm, and the pitch T of the projections 51 is preferably 0.01 mm to 0.1 mm. A plurality of projections 51 may be continuously formed with a pitch T of 0 mm. Preferably, the projecting length h of each of the projections 51, which is a length by which the projection 51 projects into a space between adjacent turns of the strand 6a, is, for example, 0.1H to 0.9H, where H is the height of the projection 51.

In the wire 1 according to the present disclosure, the projections 51 are formed on the side surfaces 5a and 5b of the flat portion 5 extending in the longitudinal direction as described above. Therefore, frictional resistance acts on the projections 51, which are in contact with the strand 6a, when the proximal portion of the wire 1 is rotated to pass the wire 1 through a meandering or branched blood vessel or through a stenotic portion. Accordingly, twisting of the flat portion 5 can be suppressed. As a result, the distal portion of the wire 1 can be directed toward an intended direction, because a torque applied to the proximal portion of the wire 1 can be effectively transmitted to the distal portion. Thus, the torque transmission ability of the wire 1 can be improved.

Next, modifications of the wire 1 according to the first embodiment of the present disclosure will be described.

As illustrated in FIG. 1, in the wire 1, preferably, the core member 2A and the coil member 6 are fixed to each other at a plurality of positions, although it is sufficient that the core member 2A and the coil member 6 be fixed to each other at one position in the distal portion.

For example, as illustrated in FIG. 1, in the wire 1, the distal portion of the core member 2A (the flat portion 5) and the distal portion of the coil member 6 (the second coil member 62) are fixed to each other by using the fixing material (fixing portion) 72; an intermediate portion of the core member 2A (including a proximal portion of the transition portion 4, the small-diameter portion 35, and a distal portion of the second tapered portion 34) and an intermediate portion of the coil member 6 (the boundary portion 63) are fixed to each other by using a fixing material (fixing portion) 73; and another intermediate portion of the core member 2A (including a proximal portion of the medium-diameter portion 33 and a distal portion of the first tapered portion 32) and the proximal portion of the coil member 6 (the first coil member 61) are fixed to each other by using a fixing material (fixing portion) 71.

Here, each of the fixing materials (fixing portions) 71, 72, and 73 is a solder (brazing alloy) or an adhesive. A method of fixing the core member 2A and the coil member 6 to each other is not limited to a method using the fixing materials (fixing portions) 71, 72, and 73 as described above. The fixing portions 71, 72, and 73 may be, for example, formed by welding.

As illustrated in FIG. 1, preferably, the wire 1 can include a resin coating 8, which covers the surface of at least the distal portion of the coil member 6.

To be specific, preferably, the resin coating 8 covers a part or the entirety of the surface of the wire 1, that is, the entire surface of the second coil member 62, the entire surface of the coil member 6 (including the first coil member 61, the boundary portion 63, and the second coil member 62), or the entire surfaces of the coil member 6 and a proximal portion of the core member 2A.

Preferably, in accordance with an exemplary embodiment, the resin coating 8 is made of a resin material, such as a fluororesin, a maleic anhydride polymer, or polyurethane. Preferably, the resin coating 8 has a thickness of, for example, 0.001 mm to 0.05 mm. By covering the wire 1 with the resin coating 8, the frictional resistance (sliding resistance) of the wire 1 is reduced, and therefore the operability of the wire 1 in a blood vessel can be improved.

Next, a method of using the guidewire according to the present disclosure will be described by using PTCA as an example.

The distal end of the guidewire is made to protrude from the distal end of a guiding catheter. In this state, the guidewire and the guiding catheter are inserted into the femoral artery by using the Seldinger technique, and inserted into the right coronary artery via the aorta, the aortic arch, and the ostium of the right coronary artery. While retaining the guiding catheter at the position of the ostium of the right coronary artery, only the guidewire is advanced in the right coronary artery to pass the guidewire through a stenotic portion of a blood vessel. Then, the guidewire is stopped at a position at which the distal end of the guidewire has passed beyond the stenotic portion of the blood vessel. Thus, a path for a balloon catheter for expanding the stenotic portion is formed.

Next, the distal end of the balloon catheter, which has been inserted from the proximal portion of the guidewire, is made to protrude from the distal end of the guiding catheter. The balloon catheter is advanced further along the guidewire, inserted into the right coronary artery from the ostium of the right coronary artery, and stopped when the balloon of the balloon catheter reaches the position of the stenotic portion of the blood vessel.

Next, the balloon is inflated by injecting a fluid for inflating the balloon from the proximal portion of the balloon catheter, thereby expanding the stenotic portion of the blood vessel. By doing so, a deposit of cholesterol and other substances adhering to the stenotic portion of the blood vessel is physically expanded, so that obstruction of blood flow can be removed.

The balloon is deflated by draining the fluid for inflating the balloon from the inside of the balloon. Next, the balloon catheter, the guidewire, and the guiding catheter are extracted from the blood vessel by moving the balloon catheter and the guidewire together toward the proximal end. This completes the PTCA operation.

The detailed description above describes a guidewire that is used to guide a catheter into a body lumen, in particular, into a blood vessel. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A guidewire comprising:
a core member that is an elongated member having flexibility; and
a coil member that is disposed so as to cover a distal portion of the core member and that is formed by helically winding a strand, the coil member being fixed to the core member in a distal portion of the guidewire,
wherein the core member includes a body portion in a proximal portion of the core member and a flat portion in the distal portion of the core member, and
wherein at least one projection is formed on at least one of two side surfaces of the flat portion extending in a longitudinal direction, the at least one projection projecting into a space between adjacent turns of the strand and being in contact with the strand.

2. The guidewire according to claim 1, wherein a longitudinal sectional shape of the projection is substantially triangular.

3. The guidewire according to claim 1, wherein a plurality of the projections are formed on each of the two side surfaces of the flat portion extending in the longitudinal direction, and a width of the flat portion including the projections is larger than an inside diameter of the coil member.

4. The guidewire according to claim 2, wherein a plurality of the projections are formed on each of the two side surfaces of the flat portion extending in the longitudinal direction, and a width of the flat portion including the projections is larger than an inside diameter of the coil member.

5. The guidewire according to claim 1, wherein the body portion comprises:
a large-diameter portion having a uniform outside diameter, a first tapered portion having an outside diameter that gradually decreases toward a distal end, an medium-diameter portion having a uniform outside diameter, a second tapered portion having an outside diameter that gradually decreases toward the distal end, and a small-diameter portion having a uniform outside diameter.

6. The guidewire according to claim 1, comprising:
a transition portion configured to connect a distal end of the body portion to a proximal end of the flat portion, the transition portion having a cross-sectional shape, which changes from a circular cross-sectional shape to a rectangular cross-sectional shape.

7. The guidewire according to claim 1, comprising:
a resin coating configured to cover at least a distal portion of the coil member.

8. The guidewire according to claim 1, wherein the at least one projection comprises a plurality of projections along an entire length of the at least one of two side surfaces of the flat portion.

9. The guidewire according to claim 8, wherein a longitudinal sectional shape of each of the plurality of projections is a triangular shape.

10. The guidewire according to claim 1, wherein the strand is made of a metal material.

11. The guidewire according to claim 10, wherein the metal material is stainless steel or a Pt—Ni alloy.

12. A guidewire comprising:
a core member that is an elongated member having flexibility;
a coil member configured to cover a distal portion of the core member and that is formed by helically winding a strand, the coil member being fixed to the core member in a distal portion of the guidewire, wherein the core member includes a body portion in a proximal portion of the core member and a flat portion in the distal portion of the core member; and
a plurality of projections formed on each side surface of the flat portion extending in the longitudinal direction, the plurality of projections projecting into a space between adjacent turns of the strand and being in contact with the strand.

13. The guidewire according to claim 12, wherein a longitudinal sectional shape of each of the projections is substantially triangular.

14. The guidewire according to claim 12, wherein a width of the flat portion including the projections is larger than an inside diameter of the coil member.

15. The guidewire according to claim 12, wherein the body portion comprises:
a large-diameter portion having a uniform outside diameter, a first tapered portion having an outside diameter that gradually decreases toward a distal end, an medium-diameter portion having a uniform outside diameter, a second tapered portion having an outside diameter that gradually decreases toward the distal end, and a small-diameter portion having a uniform outside diameter.

16. The guidewire according to claim 12, comprising:
a transition portion configured to connect a distal end of the body portion to a proximal end of the flat portion, the transition portion having a cross-sectional shape, which changes from a circular cross-sectional shape to a rectangular cross-sectional shape.

17. The guidewire according to claim 12, comprising:
a resin coating configured to cover at least a distal portion of the coil member.

18. The guidewire according to claim 12, wherein a longitudinal sectional shape of each of the plurality of projections is a triangular shape.

19. The guidewire according to claim 12, wherein the strand is made of a metal material.

20. The guidewire according to claim 19, wherein the metal material is stainless steel or a Pt—Ni alloy.
